(19) United States
(12) Patent Application Publication
(10) Pub. No.: US 2022/0296338 A1
(43) Pub. Date: Sep. 22, 2022

(54) DENTAL HANDPIECE STRUCTURE

(71) Applicant: Chih-I Tsai, New Taipei City (TW)

(72) Inventor: Chih-I Tsai, New Taipei City (TW)

(21) Appl. No.: 17/202,880

(22) Filed: Mar. 16, 2021

(51) Int. Cl. A61C 1/08; A61B 1/06; A61B 1/24; A61B 1/07

(52) U.S. Cl. CPC A61C 1/088; A61B 1/0676; A61B 1/24; A61B 1/07

(57) ABSTRACT

A dental handpiece structure has a head and a handle. A mounting passage is formed through the head. A positioning recess is formed in a handle end and connected to the mounting passage. A curved narrow tip is mounted to a tip end of the head. The mounting passage extends toward the narrow tip. A water outlet is formed in a proximal end of the narrow tip and is adjacent to the mounting passage. A light guiding strip is disposed in the mounting passage. The handle has a light source corresponding in position to the light guiding strip. As a result, light emitted by the light source is concentrated on the narrow tip, thereby improving visibility.

### BACKGROUND OF THE INVENTION

### 1. Field of the Invention

The present invention relates to a dental instrument, especially to a dental handpiece with LED (Light-emitting diodes) illumination.

### 2. Description of the Prior Arts

A conventional ultrasonic dental handpiece has a dental scaling insert and a coaxial light source. The scaling insert is mounted to an end of the dental handpiece. The light source is located around the scaling insert. A water flow passage is formed through the scaling insert and forms a water outlet on an end of the scaling insert. During gingival scaling, water is sprayed toward the mouth of a patient, and the mouth of the patient is illuminated by the light source for ease of operation.

However, because the light source of the conventional dental handpiece is annular, light emitted by the light source diverges and may easily shine in the eyes of the patient and the dentist, which causes discomfort.

Furthermore, the water sprayed toward the mouth of the patient generates a water mist. The light diverging from the light source combined with the water mist blurs vision of the dentist and affects accuracy of operation.

Additionally, a handle and a connecting portion of the conventional dental handpiece are cylindrical, making the dental handpiece slippery when the dentist rotates the dental handpiece with fingertips.

To overcome the shortcomings, the present invention provides a dental handpiece structure to mitigate or obviate the aforementioned problems.

### SUMMARY OF THE INVENTION

The main objective of the present invention is to provide a dental handpiece structure having a positioning recess and a light guiding strip in its head such that light emitted by a light source in a handle is projected in a concentrated manner.

The dental handpiece structure has a head and a handle. The head has a connecting portion, a connecting rod, a narrow tip, and a light guiding strip. The connecting portion has a tip end, a handle end, a mounting passage, and a positioning recess. The tip end and the handle end are disposed opposite each other. The mounting passage is formed through the tip end and the handle end. The positioning recess is formed in the handle end and is connected to the mounting passage. The connecting rod protrudes from the handle end of the connecting portion. The narrow tip is curved and is mounted to the tip end of the connecting portion. The narrow tip has a distal end, a proximal end, a tip point, and a water outlet. The tip point is located on the distal end of the narrow tip. The water outlet is formed in the proximal end of the narrow tip and is adjacent to the mounting passage. The light guiding strip is disposed in the mounting passage. An imaginary center line of the mounting passage extends toward the tip point of the narrow tip. The handle has a first end, a second end, a connecting recess, a light source recess, and a light source. The connecting recess is formed in the first end and is located around the connecting rod of the head. The light source recess is formed in the first end and corresponds in position to the light guiding strip. The light source is disposed in the light source recess. Light emitted by the light source travels through the light guiding strip such that the light emitted by the light source is projected toward the tip point of the narrow tip

The advantage of the present invention is that light emitted by the light source travels through the light guiding strip such that the light is projected from the mounting passage and concentrated on the tip point of the curved portion. As a result, visibility of key areas is improved by effective illumination, and blurring caused by water mist when the water outlet sprays water can be mitigated.

Additionally, because a front end portion of the light source is mounted in the positioning recess, the present invention prevents the light emitted by the light source from leaking through a gap between a handle end surface of the head and a first end surface of the handle, and therefore prevents the light of the light source from shining in the eyes of the patient and the dentist, which causes discomfort.

Other objectives, advantages and novel features of the invention will become more apparent from the following detailed description when taken in conjunction with the accompanying drawings.

### BRIEF DESCRIPTION OF THE DRAWINGS

FIG. 1 is a perspective view of a dental handpiece structure in accordance with the present invention;

FIG. 2 is an exploded view of the dental handpiece structure in FIG. 1;

FIG. 3 is a partial sectional view of the dental handpiece structure in

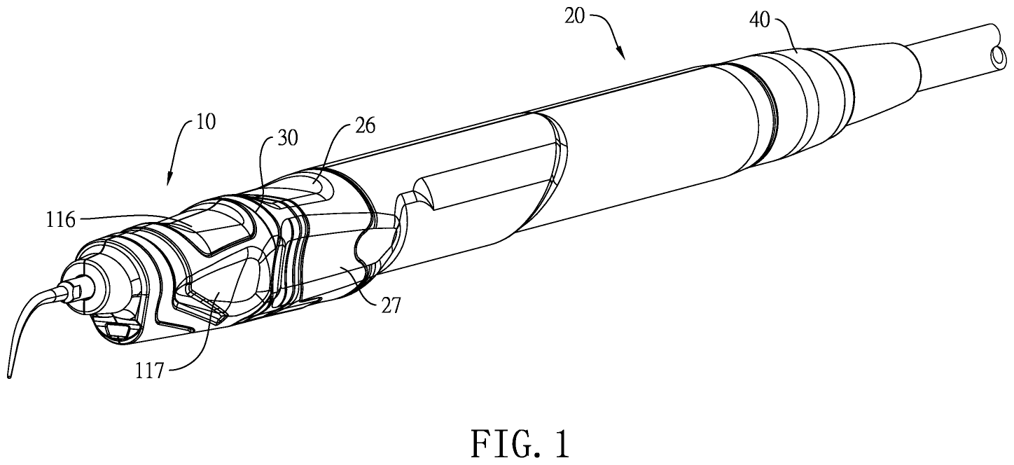

FIG. 1;

FIG. 4 is another partial sectional view of the dental handpiece structure in FIG. 1;

FIG. 5 is a sectional view of a narrow tip of the dental handpiece structure in FIG. 1;

FIG. 6 is a perspective view of the narrow tip of the dental handpiece structure in FIG. 5;

FIG. 7A is a perspective view of the dental handpiece structure, showing the dental handpiece structure in a dissembled status;

FIG. 7B is an enlarged view of FIG. 7A;

FIG. 8A is a perspective view of the dental handpiece structure, showing the dental handpiece structure in an assembled status;

FIG. 8B is an enlarged view of FIG. 8A; and

FIG. 9 is a perspective view of the dental handpiece structure in FIG. 1, showing an operation status of the dental handpiece structure.

### DETAILED DESCRIPTION OF THE PREFERRED EMBODIMENTS

With reference to FIG. 1, a dental handpiece structure in accordance with the present invention comprises a head 10, a handle 20, multiple anti-slip jackets 30 and a tail cover 40.

With reference to FIGS. 1 to 3, the head 10 has a connecting portion 11, a connecting rod 12, a narrow tip 13 and a light guiding strip 14.

The connecting portion 11 is elongated and has a tip end, a handle end, a tip end surface 111 located at the tip end, a handle end surface 112 located at the handle end, a front gripping surface 113, a mounting passage 114, a positioning recess 115, a front upper gripping portion 116 and two front lateral gripping portions 117.

The tip end and the handle end are disposed opposite each other. The front gripping surface 113 is located between the tip end surface 111 and the handle end surface 112. The mounting passage 114 is formed through the tip end surface 111 and the handle end surface 112 and extends along an axial direction of the connecting portion 11. The positioning recess 115 is formed in the handle end surface 112 and connected to the mounting passage 114.

The front upper gripping portion 116 is formed in the front gripping surface 113 of the head 10. The two front lateral gripping portions 117 are formed in the front gripping surface 113. The front upper gripping portion 116 and the two front lateral gripping portions 117 are annularly disposed around the front gripping surface 113. In preferred embodiment, the front upper gripping portion 116 is recessed to correspond in contour to a user's forefinger. The two front lateral gripping portions 117 correspond in contour to the user's thumb and middle finger respectively.

With reference to FIG. 4, a cross section of the connecting portion 11 is substantially elliptical triangular to be ergonomic, but the connecting portion 11 is not limited thereto as long as shapes and positions of the front upper gripping portion 116 and the front lateral gripping portions 117 enable the dentist to stably hold the present invention by hand.

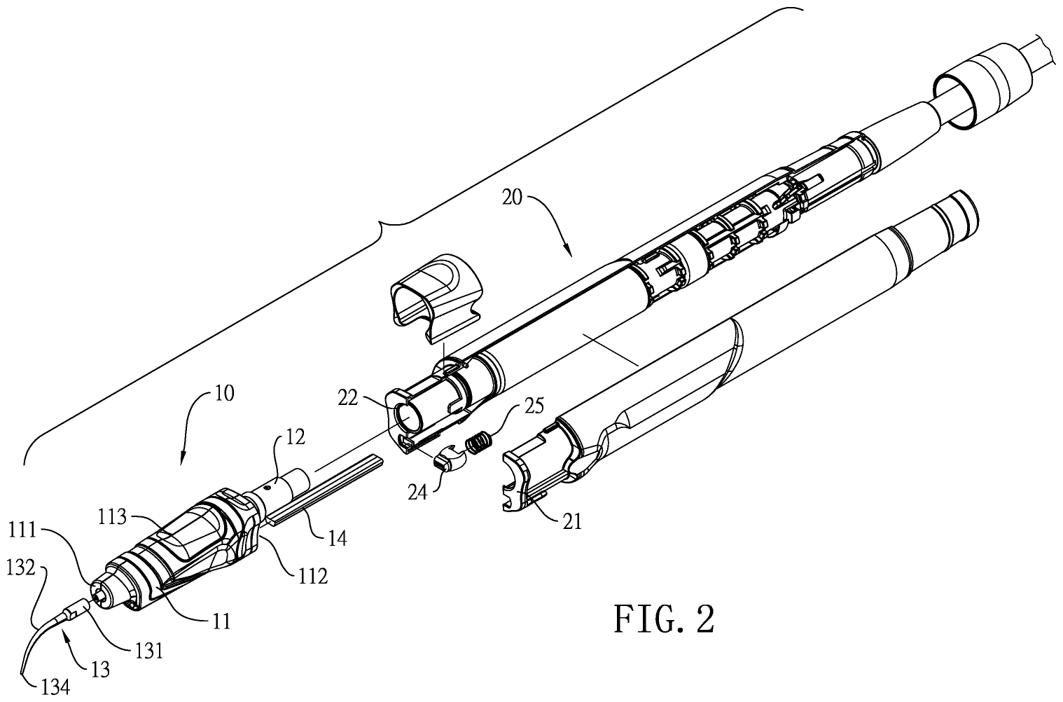

With reference to FIGS. 2, 3, 5, and 6, the connecting rod 12 protrudes from the handle end surface 112 of the connecting portion 11. An alloy plate (not shown in figures) is connected to an end, which is opposite to the connecting portion 11, of the connecting rod 12. The alloy plate is, but not limited to, nickel alloy. The alloy plate expands and contracts in response to magnetic field. Dimensional change of the alloy plate is amplified by the connecting rod 12 and the narrow tip 13 to generate ultrasonic vibration at a frequency greater than 20 kHz. The alloy plate is a prior art of standard dental handpiece; therefore detailed description of the alloy plate is omitted.

The narrow tip 13 is curved and mounted to the tip end of the connecting portion 11. The narrow tip 13 has a distal end, a proximal end, a base 131 and a curved portion 132. A connecting recess is formed in an end surface of the base 131. A water outlet 133 is formed through the base 131 and fluidly communicates with the connecting recess of the base 131. The connecting recess of the base 131 is fixed to the connecting portion 11. A tip point 134 is formed on an end of the curved portion 132. A water guiding groove 135 is formed in an outer surface of the curved portion 132. The water guiding groove 135 extends from the water outlet 133 toward the tip point 134.

The base 131 is fixed to the tip end surface 111 of the connecting portion 11. The water outlet 133 is adjacent to the mounting passage 114. The light guiding strip 14 is disposed in the mounting passage 114. An imaginary center line of the mounting passage 114 extends toward the tip point 134 of the narrow tip 13 such that light emitted via the light guiding strip 14 is aimed at the tip point 134 of the narrow tip 13. The connecting rod 12 is a prior art of standard dental handpiece; therefore detailed description of the alloy plate is omitted.

With reference to FIGS. 1 to 3, the handle 20 has a first end, a second end, a first end surface 21, a connecting recess 22, a light source recess 23, a light source 24, a resilient element 25, a rear upper gripping portion 26, and two rear lateral gripping portions 27. The first end surface 21 is located at the first end of the handle 20. The connecting recess 22 is formed in the first end surface 21 and corresponds in position to the connecting rod 12. The light source recess 23 is formed in the first end surface 21. An inner diameter of an opening of the light source recess 23 is smaller than an inner diameter of a bottom of the light source recess 23; in other words, the opening of the light source recess 23 tapers. The opening of the light source recess 23 corresponds in position to the light guiding strip 14.

The light source 24 has a front end portion 241 and a rear end portion 242. The front end portion 241 and the rear end portion 242 are each disposed on a respective one of two opposite ends of the light source 24. A periphery of the front end portion 241 is chamfered. An outer diameter of the front end portion 241 is smaller than the inner diameter of the opening of the light source recess 23 and is smaller than an inner diameter of an opening of the positioning recess 115. An outer diameter of the rear end portion 242 is larger than the inner diameter of the opening of the light source recess 23.

The light source 24 and the resilient element 25 are disposed in the light source recess 23. One end of the resilient element 25 abuts against the bottom of the light source recess 23, and another end of the resilient element 25 abuts against the rear end portion 242 of the light source. The front end portion 241 of the light source 24 is attached to an inner surface of the light source recess 23 and is adjacent to the opening of the light source recess 23. The light source 24 is movable in the light source recess 23 and is movable along an axial direction of the handle 20. The connecting rod 12 of the head 10 is selectively mounted in the connecting recess 22.

In the preferred embodiment, the light source 24 is an LED light assembly, and the handle 20 is made up of two casings that are connected together via ultrasonic welding. Before welding the two casings, precision components are disposed in the handle 20 and thorough visual checks are performed to ensure. The two casings are welded together after the precision components have been checked, thereby improving product quality and effectiveness, but assembly of the handle 20 is not limited thereto.

The rear upper gripping portion 26 is formed in the front gripping surface 113. The two rear lateral gripping portions 27 are formed in the front gripping surface 113. The rear upper gripping portion 26 and the two rear lateral gripping portions 27 are annularly disposed around the front gripping surface 113. In the preferred embodiment, the rear upper gripping portion 26 corresponds in position and contour to the user's forefinger. The two rear lateral gripping portions 27 correspond in position and contour with the user's thumb and middle finger respectively.

With reference to FIGS. 1 and 2, each one of the anti-slip jackets 30 is an annular panel. The multiple anti-slip jackets 30 include a first anti-slip jacket and a second anti-slip jacket. The first anti-slip jacket is located around and fixed to the connecting portion 11. The first anti-slip jacket is adjacent to the front upper gripping portion 116 and the two front lateral gripping portions 117. The second anti-slip jacket is located around and fixed to the handle 20. The second anti-slip jacket is adjacent to the rear upper gripping portion 26 and the two rear lateral gripping portions 27. In the preferred embodiment, each one of the anti-slip jacket 30 is a flexible silicone sleeve. A thread is formed on outer surface of each one of the anti-slip jackets 30, but not limited thereto. Shapes and sizes of the anti-slip jackets 30 can be altered according to needs.

The tail cover 40 is an annular panel located around the second end of the handle 20. In the preferred embodiment, the second end of the handle 20 is connected to a cable which provides power for the light source 24 and water for the narrow tip 13. The cable is a prior art of standard dental handpiece; therefore detailed description of the cable structure is omitted. The tail cover 40 is adjacent to a position where the handle 20 is connected to the cable. The tail cover 40 is made of metal, such as stainless steel, but the tail cover 40 is not limited thereto. Material and position of the tail cover 40 can be altered according to needs as long as the tail cover 40 improves structural strength.

With reference to FIG. 2, when the present invention is in use, the head 10 can be rotationally assembled with the handle 20 by inserting the connecting rod 12 into the connecting recess 22. Assembly method is a prior art; therefore detailed description is omitted.

With reference to FIG. 7A and FIG. 7B, in the process of rotationally assembling the head 10 with the handle 20, the front end portion 241 of the light source 24 is abutted by a periphery of the handle end surface 112 of the connecting portion 11 such that the light source 24 is moved toward the bottom of the light source recess 23 and compresses the resilient element 25.

With reference to FIG. 8A and FIG. 8B, after the head 10 is rotationally assembled to the handle 20, the opening of the light source recess 23 is aligned with the opening of the positioning recess 115 (meaning the handle end surface 112 closely adjacent to the first end surface 21), and the light source 24 is moved toward the positioning recess 115 by the resilient element 25 until the front end portion 241 of the light source 24 is located in the positioning recess 115. In other words, an end, which is adjacent to the opening of the light source recess 23, of the rear end portion 242 of the light source 24 abuts against an inner surface, which is adjacent to the opening of the light source recess 23, of the light source recess 23.

With reference to FIGS. 8A, 8B, and 9, light emitted by the light source 24 travels through the light guiding strip 14 such that the light is projected from the mounting passage 114 and concentrated on the tip point 134 of the curved portion 132. As a result, visibility of key areas is improved by effective illumination, and blurring caused by water mist when the water outlet 133 sprays water can be mitigated.

Additionally, because the front end portion 241 of the light source 24 is mounted in the positioning recess 115, the present invention prevents the light emitted by the light source 24 from leaking through a gap between the handle end surface 112 of the head 10 and the first end surface 21 of the handle 20, and therefore prevents the light of the light source 24 from shining in the eyes of the patient and the dentist and causing discomfort.

With reference to FIG. 2 and FIG. 4, because the cross section of the connecting portion 11 is substantially elliptical triangular in a position where the dentist holds the dental handpiece structure, the dentist is able to turn the present invention accurately with ease, improving delicacy of operations that requires fine movements such as gingival scaling.

Furthermore, the front upper gripping portion 116, the two front lateral gripping portions 117, the rear upper gripping portion 26, and the two rear lateral gripping portions 27 allow the dentist to choose a proper holding gesture according to needs, thereby being more ergonomic.

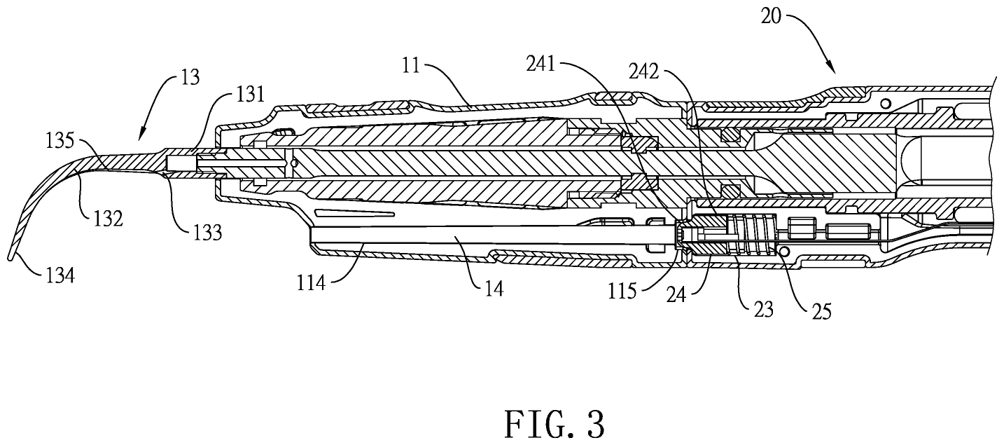

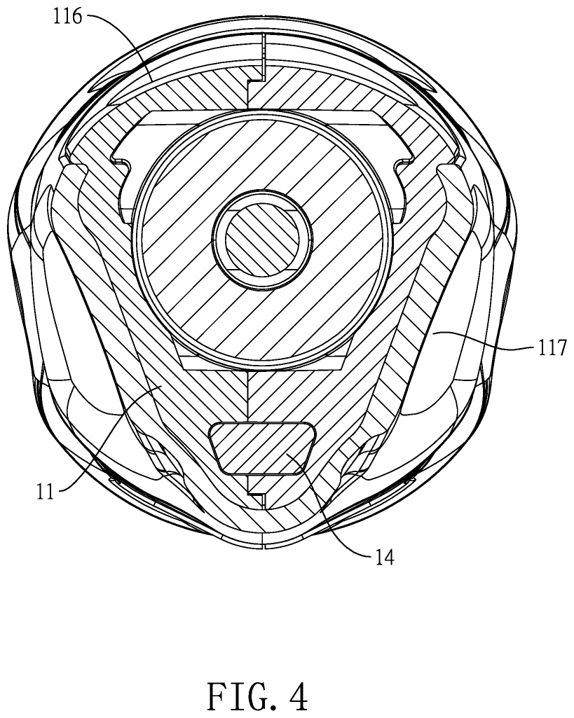

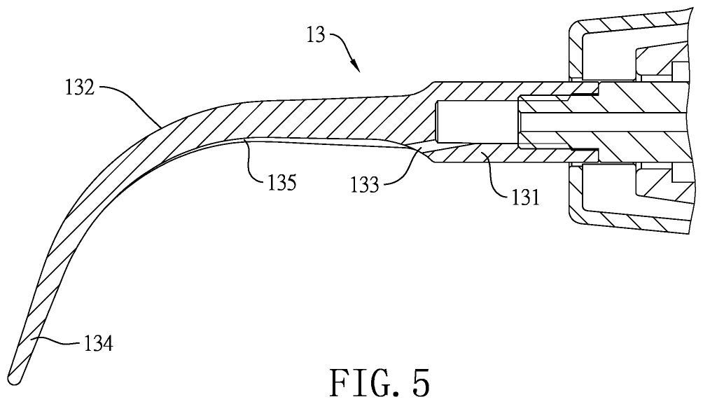

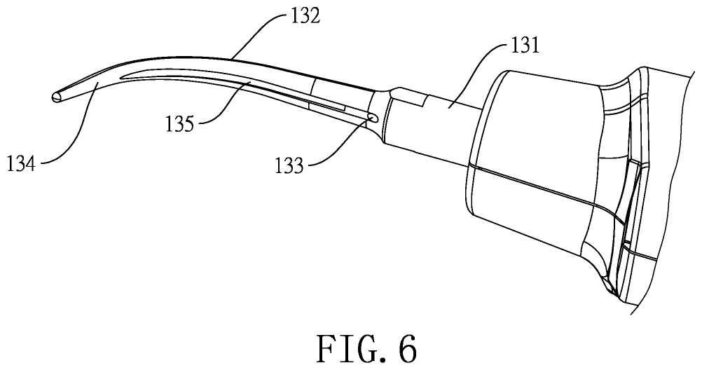

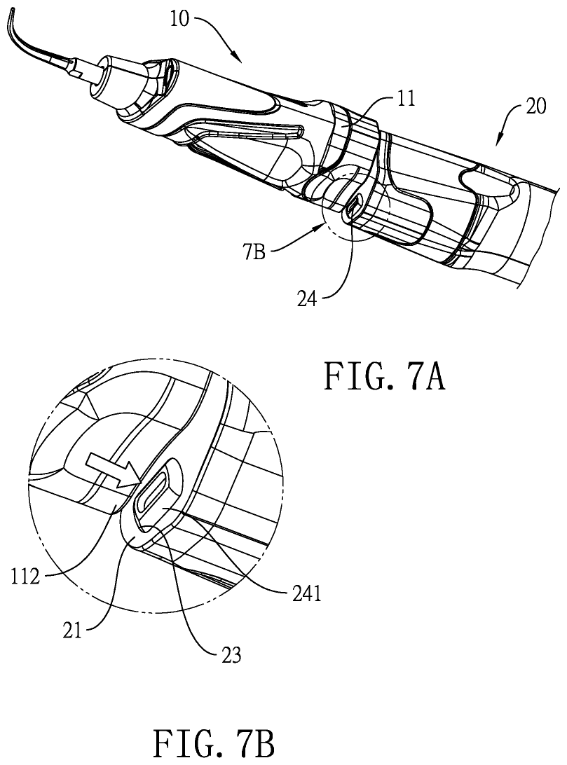

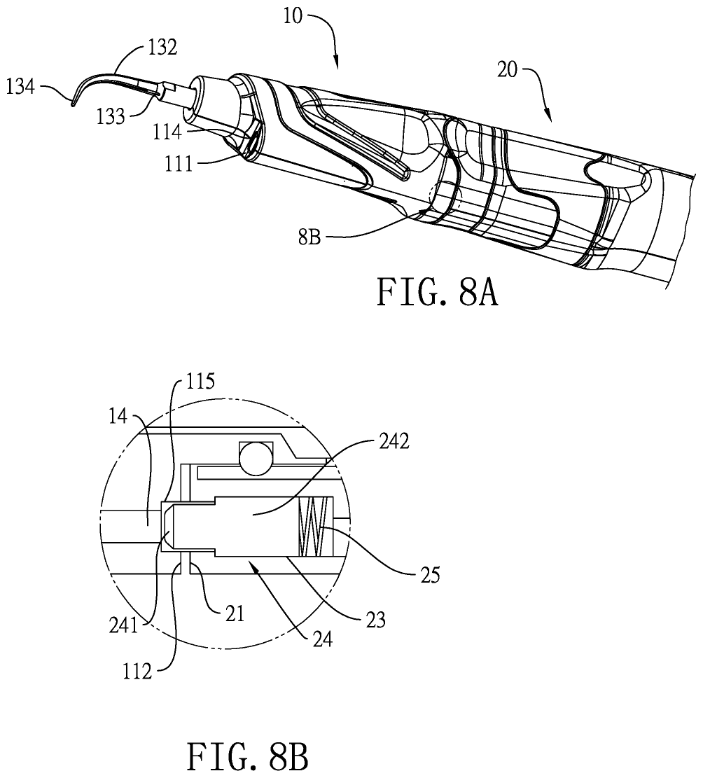

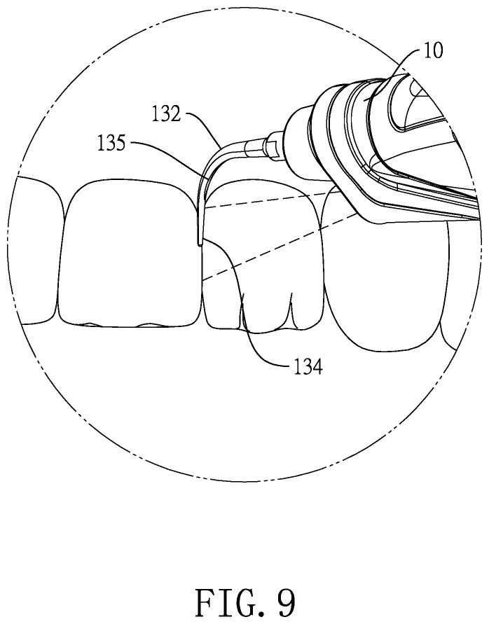

With reference to FIG. 3, because the water outlet 133 of the narrow tip 13 is formed in the base 131, structural strength of the curved portion 132 is improved in comparison with conventional narrow tips which have the water outlets formed in the curved portions. The water guiding groove 135 formed in the curved portion 132 guides the water sprayed from the water outlet 133 to the tip point 134 to cool down the narrow tip 13 and to prevent water from splashing.

With reference to FIG. 1, by mounting the anti-slip jackets 30 and forming threads on the anti-slip jackets 30, the dentist is more capable of holding the present invention stably.

By mounting the tail cover 40, the present invention prevents the second end of the handle 20 from breaking due to constant bending and pulling.

Even though numerous characteristics and advantages of the present invention have been set forth in the foregoing description, together with details of the structure and features of the invention, the disclosure is illustrative only. Changes may be made in the details, especially in matters of shape, size, and arrangement of parts within the principles of the invention to the full extent indicated by the broad general meaning of the terms in which the appended claims are expressed.

What is claimed is:

1. A dental handpiece structure comprising:
a head having:
a connecting portion having:
a tip end and a handle end disposed opposite each other;
a mounting passage formed through the tip end and the handle end; and
a positioning recess formed in the handle end and connected to the mounting passage;
a connecting rod protruding from the handle end of the connecting portion;
a narrow tip being curved and mounted to the tip end of the connecting portion; the narrow tip having:
a distal end and a proximal end;
a tip point located on the distal end of the narrow tip; and
a water outlet formed in the proximal end of the narrow tip and adjacent to the mounting passage; and
a light guiding strip disposed in the mounting passage; an imaginary center line of the mounting passage extending toward the tip point of the narrow tip; and
a handle having:
a first end and a second end;
a connecting recess formed in the first end and located around the connecting rod of the head;
a light source recess formed in the first end and corresponding in position to the light guiding strip; and
a light source disposed in the light source recess; light emitted by the light source traveling through the light guiding strip such that the light emitted by the light source is projected toward the tip point of the narrow tip.

2. The dental handpiece structure as claimed in claim 1, wherein
the handle has a resilient element disposed in the light source recess; one end of the resilient element abuts against a bottom of the light source recess; another end of the resilient element abuts against the light source; and
the light source is movable along an axial direction of the handle and is selectively mounted in the positioning recess.

3. The dental handpiece structure as claimed in claim 1, wherein
an opening of the light source recess corresponds in position to the light guiding strip;
the light source has:
a front end portion; a periphery of the front end portion chamfered; an outer diameter of the front end portion of the light source being smaller than an inner diameter of the opening of the light source recess and being smaller than an inner diameter of an opening of the positioning recess; the front end portion selectively attached to an inner surface of the light source recess and being adjacent to the opening of the light source recess; and
a rear end portion; an outer diameter of the rear end portion of the light source being larger than the inner diameter of the opening of the light source recess.

4. The dental handpiece structure as claimed in claim 3, wherein
the head has
a front upper gripping portion formed in an outer surface of the head; and
two front lateral gripping portions formed in the outer surface of the head; the front upper gripping portion and the two front lateral gripping portions annularly disposed around the head;
a cross section of the head where the front upper gripping portion and the two front lateral gripping portions are located is elliptical triangular.

5. The dental handpiece structure as claimed in claim 4, wherein
the handle has
a rear upper gripping portion formed in the outer surface of the head; and
two rear lateral gripping portions formed in the outer surface of the head; the rear upper gripping portion and the two rear lateral gripping portions annularly disposed around the handle.

6. The dental handpiece structure as claimed in claim 5, wherein
the narrow tip has:
a base having a connecting recess formed in an end surface thereof; the connecting recess assembled with the connecting portion; the water outlet formed through the base and fluidly communicating with the connecting recess of the base;
a curved portion; the tip point located on one end of the curved portion; another end, which is opposite to the tip point, of the curved portion connected to the base; and
a water guiding groove formed in an outer surface of the curved portion; the water guiding groove extending from the water outlet toward the tip point.

7. The dental handpiece structure as claimed in claim 6 comprising a tail cover; the tail cover being an annular panel located around the second end of the handle.

8. The dental handpiece structure as claimed in claim 7 comprising multiple anti-slip jackets made of anti-slip material; the multiple anti-slip jackets including a first anti-slip jacket and a second anti-slip jacket;
the first anti-slip jacket located around and fixed to the connecting portion; the first anti-slip jacket being adjacent to the front upper gripping portion and the two front lateral gripping portions;
the second anti-slip jacket located around and fixed to the handle; the second anti-slip jacket being adjacent to the rear upper gripping portion and the two rear lateral gripping portions.
